(19) United States
(12) Patent Application Publication
(10) Pub. No.: US 2026/0216473 A1
(43) Pub. Date: Jul. 30, 2026

(54) CATHETERISATION DEVICE

(71) Applicant: Sulacare AS, Mo i Rana (NO)

(72) Inventors: Marie Steinfjell, Mo i Rana (NO); Erik Pavels Petersen, Porsgrunn (NO); Preben Hjallum, Porsgrunn (NO)

(73) Assignee: Sulacare AS, Mo i Rana (NO)

(21) Appl. No.: 19/141,819

(22) PCT Filed: Jun. 27, 2024
(86) PCT No.: PCT/EP2023/087500

§ 371 Date: Jun. 20, 2025

(30) Foreign Application Priority Data
Dec. 22, 2022 (GB) 2219581.2

(51) Int. Cl. A61M 25/00

(52) U.S. Cl. CPC A61M 25/0017; A61M 2205/0216; A61M 2210/1092; A61M 2210/1475; A61M 2210/1483

(57) ABSTRACT

A device (10) for spreading the labia of a patient, the device (10) comprising: a body portion (12) comprising a proximal section (32) and a distal section (22), wherein the distal section (22) of the body portion (12) is for insertion into the vagina of the patient; and first and second wing portions (14, 16) extending from the proximal section (32) of the body portion (12), the first and second wing portions (14, 16) being configured to, in use, push against the labia and thereby spread them; wherein the first and second wing portions (14, 16) are rotatable about the body portion (12) such that the first and second wing portions (14, 16) can rotate toward one another during insertion between the labia.

The present invention relates to a device for spreading the labia of a patient. Methods for use of the device to aid catherization are also described.

There are various medical procedures for a female patient that require access to body parts that lie underneath the labia of the patient. For example, catheterisation requires insertion of a catheter into the urethra of a patient in order to remove urine from the bladder. In female patients, the urethra is typically hidden beneath the labia of the patient, and so the labia must first be moved before the urethra can be accessed. This spreading of the labia is usually done manually using one hand of a medical practitioner. However, in order to ensure that the area is kept completely sterile during the procedure, the medical practitioner must retain their hand in the position for spreading the labia throughout the entire procedure. As a result, the medical practitioner will have only one hand free to perform the actual procedure, and the presence of a second medical practitioner is needed in order to carry out any actions that the first medical practitioner cannot do using only one hand. For example, the second medical practitioner may be needed to hand over instruments to the first medical practitioner during the procedure. This results in the procedure becoming both time and labour intensive, as the two practitioners must co-ordinate in order to complete the procedure.

Such manual spreading of the labia using hands has further disadvantages due to the strict sterilization requirements associated with medical procedures such as catheterisation. For example, if the first medical practitioner loses grip of the labia and the labia return to their natural position, the area is no longer considered sterile and the entire process must restart. This lengthens the time needed for the procedure and produces considerable waste. Furthermore, such procedures naturally introduce a risk of infection, and so the more times a procedure must be repeated, the greater the risk of infection becomes. The risk of injury to the patient also increases when multiple attempts are required, along with the level of discomfort for the patient.

There is therefore a need for an improved, ‘hands-free’ method of spreading the labia of a female patient during medical procedures.

According to a first aspect of the invention, there is provided a device for spreading the labia of a patient, the device comprising:
a body portion comprising a proximal section and a distal section, wherein the distal section of the body portion is for insertion into the vagina of the patient; and
first and second wing portions extending from the proximal section of the body portion, the first and second wing portions being configured to, in use, push against the labia and thereby spread them;

wherein the first and second wing portions are rotatable about the body portion such that the first and second wing portions can rotate toward one another during insertion between the labia.

The device of the first aspect allows a user to have easy access to the body parts that are usually hidden, at least in part, by the labia without the user having to manually hold the labia apart themselves. That is, the device of the first aspect enables the user to have free use of their hands during interaction with the body parts usually covered by the labia, as the wing portions effectively replace what would typically be a user's hand holding the labia apart. Moreover, the device provides a particularly reliable and consistent method of spreading the labia, as during use it is held fixed in position via the distal section of the body portion, which is secured within the vagina.

The device is for spreading the labia of the patient, such as the labiate folds of a human patient, particularly the labia majora of the patient. The device may further be for spreading both the labia majora and the labia minora of the patient. The device may be for holding and/or moving the labia outwardly such that they move away from the urethra. The device may be for holding/spreading the labia apart at least such that the urethra of the patient is exposed. The method of use of the device may include, as discussed further below, using one hand to spread the labia before then applying the device to the patient. The manual spreading of the labia may be done in the usual way, e.g. with fingers reaching down from above, i.e. downward in a direction toward the anus.

The body portion may comprise a hollow tube. Thus, the body portion may comprise an opening extending across its length. The opening may be suitable for receiving other instruments, such as a fluid pump and/or an inflatable balloon. The body portion, or a part thereof, may comprise a flexible material for easy insertion into the vagina. Additionally, or alternatively, the body portion, or a part thereof, may comprise a rigid material and/or a rigid form for holding the wing portions securely in place. For example, the distal section of the body portion may comprise a flexible material whilst the proximal section of the body portion may comprise a rigid material. Alternatively, the body portion may be provided by a unitary body comprising the same material(s) throughout, such as by being moulded or additively manufactured from a single material, such as a polymer material as discussed below. The unitary body may also incorporate the wing portions, which thus may be formed in a single piece along with the body portion.

The proximal section of the body portion may be the section that, in use, is furthest from the patient's body, i.e. proximate the hand of the user. The distal section of the body portion may be the section that, in use, is closest to and/or within the patient's body. The proximal section may extend from one end, i.e. a proximal end, of the body portion whilst the distal section may extend from the opposite end, i.e. a distal end, of the body portion.

A proximal part of the body portion, including the distal section, may be configured to extend into the vagina by 60-100 mm, such as by about 80 mm. The distal section may be held securely in place through contact with the vaginal walls. The distal section may therefore be considered to act as securing means for the device, as the distal section may create a secure attachment between the device and the patient body during use. The wing portions of the device may, in use, span the distance between the vagina and the uppermost portion of the labia. In this way, the distal section may be further considered to act as a positioning means for the device, as the positioning of the distal section within the vagina may provide a point of reference that ensures that the wing portions are always positioned correctly relative to the labia. The wing portions may each have a length in the range 40-100 mm, such as a length of around 50-70 mm. That length may be measured via a straight line from the body portion to a tip of the wing portion. The device may be provided in differing sizes to cater for different body shapes of the patient.

The first and second wing portions may, in use, extend in a direction going upwardly from the body portion. In this context, the “upward” direction is defined relative to device during use, and so the wing portions can be considered to extend upward in a direction from the vagina of the patient towards the urethra of the patient during use. The first and second wing portions may extend from a position adjacent the vagina to a position adjacent the uppermost portion of the labia. That is, the first and second wing portions may each comprise a base adjacent the vagina, and a tip adjacent the uppermost portion of the labia. The base of a wing portion may be the end of the wing portion that is attached to the body portion, whilst the tip of a wing portion may be the opposite end of the wing portion. The wing portions may extend symmetrically about the body portion. The wing portions may for example extend upwardly by 35 to 70 mm, as well as outwardly (away from each other) by about 25-35 mm each, thus giving a width of the device that is in the range 50-70 mm, such as a width in the range 55-60 mm. This may be a width with no forces applied to the device, e.g. the open position described below.

The first and second wing portions may be flexibly attached to the body portion, thus enabling their rotational movement about the body portion; that is, the wing portions may flex at least at their base where they are attached to the body portion to enable their tips to move generally in an arc about the axis of the body portion. The first and second wing portions may comprise elastic material, such as a polymer material. Thus, the first and second wing portions may be rotatable through elastic deformation. Possible materials for the wing portions include polypropylene or polyurethane materials, for which medical grade compositions are available and would be suitable.

The body portion may comprise a slit extending from the proximal end of the body portion. The slit may be considered a “cut-out” section of the outer wall of the body portion at the proximal end. The slit may, for example, be rectangular in shape. The slit may extend to a point along the length of the body portion that is aft (i.e. further toward the distal end of the body portion) of a proximate edge of each wing portion. For example, the slit may extend to the width-wise midpoint of each wing portion. The slit may be sized and shaped such that it provides added flexibility to the device, in particular to the wing portions. The slit may therefore facilitate rotation of the wing portions.

However, the body portion need not comprise such a slit. In order to achieve the desired flexibility for the device, the device may comprise a suitably flexible material for allowing the wing portions to rotate toward one another. For example, the device may comprise polypropylene, in particular polypropylene homopolymer. The device may comprise medical-grade polypropylene, such as Lotte SJ-170M polypropylene. In particular, the wing portions may comprise polypropylene, Lotte SJ-170M polypropylene, and/or polypropylene homopolymer. Additionally or alternatively, the body portion may comprise polypropylene, Lotte SJ-170M polypropylene, and/or polypropylene homopolymer. Preferably, as discussed above, the wings and body portion of are formed as a single piece, in which case, this piece is formed of one of the materials referred to above.

The first and second wing portions may be spaced apart from one another by a wing gap. The wing gap may be the area between the inner surfaces of each wing portion, wherein the inner surface of a wing portion may be the surface that faces the body portion of the device. The wing gap may be defined through a wing distance, which may be the distance between the tips of each wing portion. At rest, the first and second wing portions may be configured to lie in an open position. The open position may be the position in which the wing distance is at its maximum. In some examples the wing distance in the open position may be in the range 25 to 40 mm, such as in the range 30 to 35 mm. The overall width to the outer curved parts of the wings may be as above in that case, e.g. in the range 50-70 mm, such as a width in the range 55-60 mm. The wing portions may be resiliently biased, e.g. by their elasticity, toward the open position. The first and second wing portions may be elastically deformable such that they rotate toward one another into an insertion position. The insertion position may be the position in which each wing portion is held during insertion between the labia. A user may exert an inward force on the wing portions and thereby elastically deform the wing portions into the insertion position. The wing distance may be at its minimum when the wing portions are in the insertion position. The wing distance may be zero when the wing portions are in the insertion position. That is, the tips of the wing portions may be in contact with one another in the insertion position, such as by curving the elastic wing portions toward one another so that their tips meet. As a result, the wing gap can be minimised during insertion of the wing portions between the labia, thus making it easier for a medical practitioner to use one hand to fit the wing portions between the labia during the initial positioning of the device, whilst the labia are spread via use of the other hand of the medical practitioner.

The wing portions may, in use, lie in a spreading position. The wing portions may be resiliently biased from the insertion position toward the spreading position, i.e. when no force is applied then the wing portions would tend to move from the insertion position toward/past the spreading position. If the device were not in use with no forces from labia, and no other forces, then the wing portions would sit stably in the open position. The spreading position of the wing portions may be the position in which, in use, the force exerted on the wing portions by the labia is equal to the elastic force exerted on the labia by the wing portions. That is, the spreading position may be the position in which the only external force acting on the wing portions is from the labia of the patient. In this way, it can be ensured that the device can spread the labia during use without continuous interference from the user. The wing distance when the wing portions are in the spreading position may be greater than the wing distance when the wing portions are in the insertion position. The spreading position may depend on the shape and size of the labia of a particular patient. For example, the spreading position may be the same as the open position for certain patients. Thus, the device may be suitable for a wide range of labia sizes and shapes without causing discomfort to the patient.

The first and second wing portions may have a curved form. In particular, the first and second wing portions may have a curved form at rest (i.e., when no external forces are acting on them). The first and second wing portions may be curved along their width and/or their length. The width of a wing portion may be the extent of the wing portion between its proximate edge (i.e. the edge closest to the proximate end of the body portion) and its distal edge (i.e. the edge closest to the distal end of the body portion). In this case there may be a thickness of the wing portion that is in the direction perpendicular to the length and width, e.g. a direction laterally across the labia, and this may also be considered as the thickness of the wing which can be seen when looking along the body section toward the patient's labia/vagina. The length of a wing portion may be the extent of the wing portion between the base and the tip. Thus, the first and second wing portions may comprise two axes of curvature. There may also be a varying thickness leading to differing curvature on the distal and proximal edges as well as on the inward/outward facing surfaces of the wing portions.

As noted above in some examples the wing portions may each have a length in the range 40-100 mm, such as a length of around 50-70 mm. The width of the wing portions, i.e. their dimension in the distal to proximal direction may be in the range 10-35 mm, such as in the range 15-25 mm. The thickness may vary along the length of the wing portions, e.g. in a curve designed to conform to the shape of the labia. The thickness of the wing portions, i.e. the thickness dimension when viewed facing the patient's labia, may be in the range 5-10 mm, such as 6-8 mm, and this may vary along the curve of the wing portions, such as to allow for varying degrees of stiffness.

The form of the wing portions may be defined relative to a vertical axis and a longitudinal axis of the device. The longitudinal axis may extend through the centre of the body portion from the proximal end to the distal end of the body portion. The vertical axis may extend perpendicularly to the longitudinal axis and may be parallel to the line extending between the vagina and the urethra during use of the device. The vertical axis may extend through the body portion at a position along the length of the body portion that corresponds to the position at which the wing portions are attached to the body portion.

The wing portions may curve towards the body portion in the vertical direction. For each wing portion, the angle between the wing portion and the vertical axis may be a positive obtuse angle at the base and may be a positive acute angle at the tip. The angle between the wing portion and the vertical axis at the midpoint of the wing portion along its length may be zero. The distance between the inner surface of the wing portion and the vertical axis may increase from the base to the lengthwise midpoint of the wing portion, and may decrease from the lengthwise midpoint of the wing portion to the tip. The inner surface of the wing portion may be curved concavely along the vertical direction, whilst the outer surface of the wing portion may be curved convexly along the vertical direction. The degree of curvature in the vertical direction may vary along the width of the wing portion or may be constant along the width of the wing portion. The degree of curvature in the vertical direction (i.e., the degree of vertical curvature) may vary along the length of the wing portion. In particular, the degree of vertical curvature may be greatest at the tip of the wing portion. The degree of vertical curvature may increase along the length of the wing portion, i.e. from the base to the tip. The wing portion may comprise a first degree of vertical curvature from the base to a transition point along the length of the wing portion, and may comprise a second degree of vertical curvature from the transition point to the tip, wherein the first degree of vertical curvature is smaller than the second degree of vertical curvature. In this way, the wing portions may more accurately conform to the shape of the labia, thereby keeping the spreading force exerted on the labia during use within a comfortable range for the patient.

The wing portions may curve away from the body portion in the longitudinal direction. For each wing portion, the angle between the wing portion and the longitudinal axis may be a positive acute angle at the proximate edge and may be a positive obtuse angle at the distal edge. The angle between the wing portion and the longitudinal axis at the midpoint of the wing portion along its width may be zero. The distance between the inner surface of the wing portion and the longitudinal axis may decrease from the proximate edge to the width-wise midpoint of the wing portion, and may increase from the width-wise midpoint of the wing portion to the distal edge. The inner surface of the wing portion may be curved convexly along the longitudinal direction, whilst the outer surface of the wing portion may be curved concavely along the longitudinal direction. The degree of curvature in the longitudinal direction may vary along the width of the wing portion and/or the length of the wing portion or may be constant along the width of the wing portion and/or the length of the wing portion. In particular, the degree of curvature in the longitudinal direction may be at its minimum at the tip and the base of the wing portion, and may be at its maximum at the lengthwise midpoint of the wing portion.

Thus, due to their curved forms, the wing portions of the device can mimic the approximate shape of the labia when in the spreading position, thereby ensuring maximum comfort for the patient during use as well as secure “hands-free” engagement with the labia.

The wing portions may each comprise a rounded tip. The wing portions may each comprise rounded edges. In this way, it may be ensured that the wing portions do not feel as though they are “cutting into” the patient during use.

The wing portions may be elastically deformable to provide increased curvature in the insertion position. That is, the curvature of the wing portions in the vertical direction and/or the longitudinal direction may increase during elastic deformation of the wing portions from the open position or the spreading position to the insertion position. The wing portions may for example transition from a generally V-shape configuration, to a generally O-shape configuration where in the O-shape there is contact at the top of the O between tips of the wing portions. With the use of an increased curvature the device may be in an optimal shape to engage with the labia when the user's hand has pre-spread the labia, i.e. in a conventional way as mentioned above. The wing portions may return elastically to their original form when released into the open position or the spreading position, such as with a change in the wing distance (wing tip spacing) from zero or close to zero in the open position to a wing distance in the range 25 to 40 mm, such as in the range 30 to 35 mm.

The device may further comprise an inflatable balloon portion attached to the distal section of the body portion. The body portion may for example extend to a total length of 90-120 mm, such as about 95 mm or 100 mm, with the distal section extending 20-40 mm (optionally 25-35 mm) from the location of the wing portions and a balloon portion extending a further 35-60 mm (optionally 40-50 mm), giving a length of 60-100 mm that is for insertion into the patient's vagina. The inflatable balloon portion may be insertable within the hollow tube of the body portion. The inflatable balloon portion may be fixedly attached to the distal section of the body portion. The inflatable balloon portion may be fixedly attached to the distal section using any suitable fixing means, such as adhesive.

The inflatable balloon portion of the device is therefore configured to be inserted into the vagina along with the distal section of the body portion. The inflatable balloon portion may be for securing the distal section of the body portion within the vagina. The inflatable balloon portion may be configured to exert pressure against the walls of the vagina when inflated, thereby securing the distal section of the body portion within the vagina. In some examples the balloon is configured to inflate to a diameter of 25-35 mm, e.g. about 30 mm. The balloon may have a length that in use extends along the vagina with this length being in the range 35-50 mm, such as a length in the range 40-45 mm. The inflatable balloon portion may be any suitable medical balloon, such as a Foley balloon. The balloon may be made of a suitable material, such as a silicone, polyurethane or PVC material. The inflatable balloon portion may be configured to be inflated whilst inserted within the vagina. Thus, the inflatable balloon portion may provide additional stability to the device and may ensure that the wing portions are securely held in the correct position during use of the device.

The device may comprise a fluid pump coupled to the inflatable balloon portion. The fluid pump may be configured to supply fluid to the inflatable balloon portion. In particular, the fluid pump may be an air pump and may be configured to supply air to the inflatable balloon portion. It will however be understood that the fluid pump may be configured to supply any suitable fluid to the inflatable balloon portion, such as water, and is not limited to an air pump. The fluid pump may be a syringe. The fluid pump may be configured to fill the inflatable balloon portion with the fluid during inflation of the inflatable balloon portion. The fluid pump may be configured to drain the inflatable balloon portion of the fluid during deflation of the inflatable balloon portion. The fluid pump may be removably insertable into the hollow tube of the body portion, particularly into the proximal section of the body portion. The fluid pump may be coupled to the inflatable balloon portion via the body portion. The device may be configured such that the fluid pump can be detached from the body portion after inflation of the inflatable balloon portion. In this way, it can be ensured that the fluid pump does not obstruct any procedures being performed on the patient.

The first and second wing portions may be attached to the body portion at a point inward from the proximal end of the body portion. As such, the proximal section of the body portion may extend past the wing portions by a certain distance. In this way, the proximal section of the body portion may provide a gripping point for a user. This may allow a user to securely hold onto the device during the initial positioning of the device on the patient.

The first and second portions may be integral with the body portion. The first and second portions and the body portion may be a unitary body. The wing portions and/or the body portion may comprise an antibacterial coating. Thus, the risk of infection from using the device may be minimised.

During catheterisation of a female patient, the labia must be spread throughout the duration of the procedure to allow clear, uninterrupted access to the urethra. The device of the first aspect may therefore be suitable for preparing a patient for catheterisation. In particular, the device may be suitable for preparing a patient for single-person catheterisation. This is because the device removes the need for the labia to be manually held apart by a person during any procedures being performed on the patient. As such, procedures such as catheterisation can be performed by a single person when the device of the first aspect is employed, whether that single person is a medical practitioner or the patient themselves.

According to a second aspect of the present invention, a method of spreading the labia of a patient using the device of the first aspect is provided, the method comprising: inserting the distal section of the body portion into the vagina of the patient;
gripping and rotating the first and second wing portions toward one another;
locating the first and second wing portions between the labia; and
releasing the first and second wing portions such that the first and second wing portions rotate away from one another and push against the labia.

The method may use a device according to the first aspect comprising any of the optional features given with respect to the device of the first aspect. The step of inserting the distal section into the vagina of the patient may occur concurrent with the steps of rotating the wing portions and locating them between the labia. Thus, the insertion of the device into the vagina may also move the wing portions into the required location between the labia.

The method may include rotating the first and second wing portions toward one another until the distance between the two wing portions is minimized. The method may include rotating the first and second wing portions toward one another until they are in contact with one another at their tips, i.e. until the distance between the tips of each wing portion is zero. The method may include, prior to insertion of the distal section, using a hand to spread the labia. This may be done in any conventional way, e.g. with fingers reaching down from above, i.e. downward in a direction toward the anus. Whilst one hand is used to spread the labia the other hand may be used to insert the device and facilitate the performance of the method above.

The method may include attaching an inflatable balloon portion to the distal section of the body portion prior to insertion of the distal section into the vagina. The method may include inserting the inflatable balloon portion into a hollow tube of the body portion. The method may include fixedly attaching the inflatable balloon portion to the distal section of the body portion, for example using adhesive.

The method may include inserting the inflatable balloon portion into the vagina of the patient with the distal section of the body portion. The method may include inflating the inflatable balloon portion to secure the distal section of the body portion within the vagina. The method may include inflating the inflatable balloon after the wing portions have been released into the spreading position. In this way, a user can ensure that the wing portions are correctly positioned before fixing the device in place. The method may include inflating the inflatable balloon portion until the inflatable balloon portion exerts a securing pressure onto the walls of the vagina.

The method may include inflating the inflatable balloon portion using a fluid pump, such as an air pump or a water pump. The method may include removably attaching the fluid pump to the proximal section of the body portion. The method may include coupling the fluid pump to the inflatable balloon portion, for example via the body portion. The method may include detaching the fluid pump from the device once the inflatable balloon portion has been inflated. The method may include using the fluid pump to deflate the inflatable balloon portion.

The proximal section of the body portion may extend past the wing portions, and the method may include gripping the proximal section of the body portion during positioning of the device.

The method may be suitable for preparing a patient for catheterisation. In particular, the method may be suitable for preparing a patient for single-person catheterisation, where a single person can perform the catheterisation without needing help from an additional person to manually hold the labia apart during said catheterisation.

The method may be suitable for performing catheterisation, and thus may further include inserting a catheter into the urethra of the patient after the first and second wing portions have been released. The method may include inserting the catheter into the urethra after the inflatable balloon has been inserted into the vagina and inflated. The method may include inserting the catheter into the bladder of the patient via the urethra. The method may include inflating an inflatable balloon of the catheter when the catheter is in the bladder, thereby securing the catheter in place. The method may include attaching the catheter to a drainage bag.

The method may include removing the catheter from the patient. Once the catheter is removed, the method may include deflating the inflatable balloon of the labia spreading device and removing the inflatable balloon portion from the vagina. Once the catheter is removed, the method may include rotating the first and second toward one another (e.g., until the distance between the two wing portions is minimized or zero), and removing the first and second wing portions from between the labia. This may allow the labia to return to their natural position once the catheterisation is finished.

According to a third aspect of the invention, there is provided a device for spreading the labia of the patient, the device comprising:
a body portion; and
first and second wing portions extending from the body portion, the first and second wing portions each being curved along both their width and their length to conform to the shape of the labia;
wherein the first and second wing portions are elastically biased to a position in which they, in use, push against the labia and thereby spread them; and

wherein the first and second wing portions are elastically deformable to an insertion position in which they are rotated about the body portion toward one another.

The device of the third aspect utilises an inward rotation movement during insertion of the wing portions between the labia. This may allow for tips of the wing portions to move toward and optionally come into contact with one another, e.g. as described above. The wing portions may also increase in curvature along their length as they are elastically deformed, e.g. with an elastic bending of the wing portion along the length thereof. The inward rotation during elastic deformation may move the wing portions into an insertion position, i.e. a position at which the device is ready to be engaged with the labia, as discussed above for the first and second aspects. Conversely, when released from this insertion position, the wing portions will rotate outwardly into a spreading position, optionally with a decrease in the degree of curvature along the length of the wing portions. This advantageously occurs automatically as a result of the elastic bias of the wing portions, such as due to the elastic properties of the material and/or shape thereof. In addition, the device will remain in the spread form when the user lets go of it and leaves it on the patient. This enables easy, hands-free spreading of the labia for the medical practitioner during any procedures which require access to the body parts usually shielded by the labia, whilst simultaneously ensuring comfort for the patient during the spreading of the labia.

The body portion of the device may comprise a proximal section and a distal section. The distal section of the body portion may be for insertion into the vagina of the patient. The first and second wing portions may extend from the proximal section of the body portion.

The device of the third aspect may comprise any of the optional features associated with the device of the first aspect.

According to a fourth aspect of the present invention, a method of spreading the labia of a patient using the device of the third aspect is provided. The method comprises: rotating the first and second wing portions toward one another; locating the first and second wing portions between the labia; and releasing the first and second wing portions such that the wing portions return to their elastically biased position.

The method may use a device comprising any of the optional features described with respect to the device of the third aspect. The method may include any of the optional steps described with reference to the method of the second aspect.

Certain embodiments will now be described by way of example only and with reference to the accompanying drawings, in which:

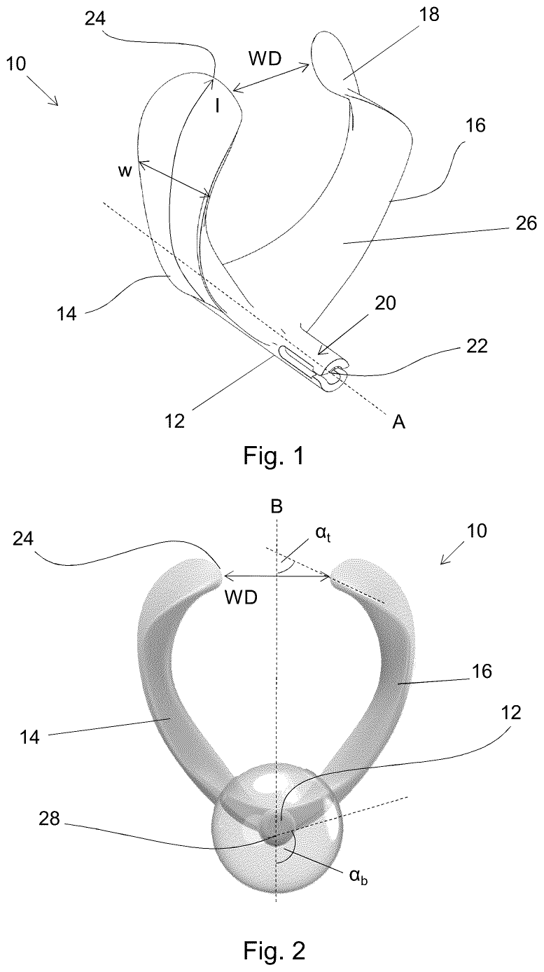

FIG. 1 is a perspective view of a medical device including a body portion and two wing portions;

FIG. 2 shows the device in an end view along with the addition of a balloon portion that is for securing the device in the vagina;

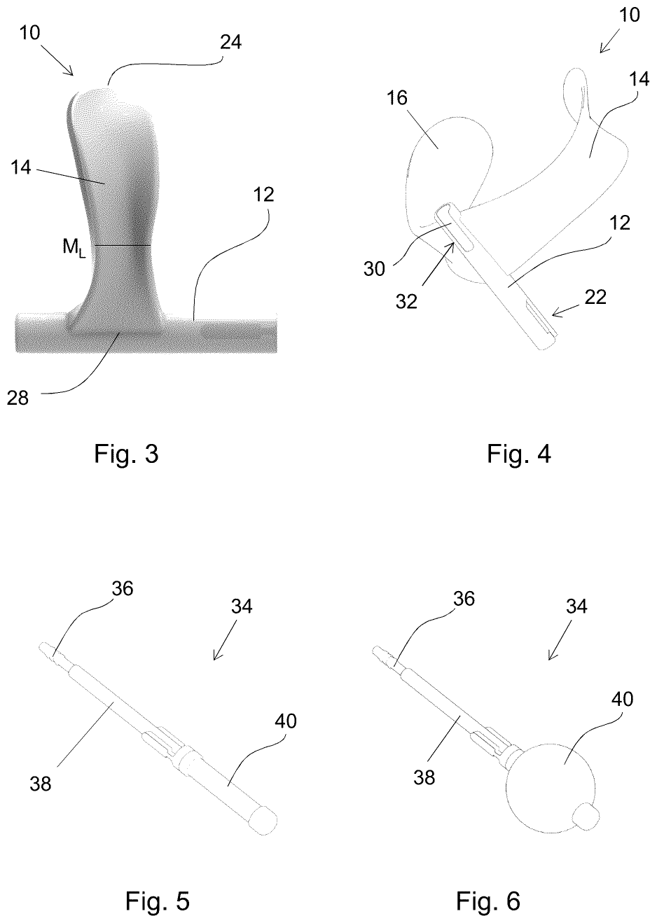

FIG. 3 is a side view of the device;

FIG. 4 shows another perspective view;

FIGS. 5 and 6 show a balloon portion for use with the device of FIG. 1, with a balloon being in uninflated and inflated configurations;

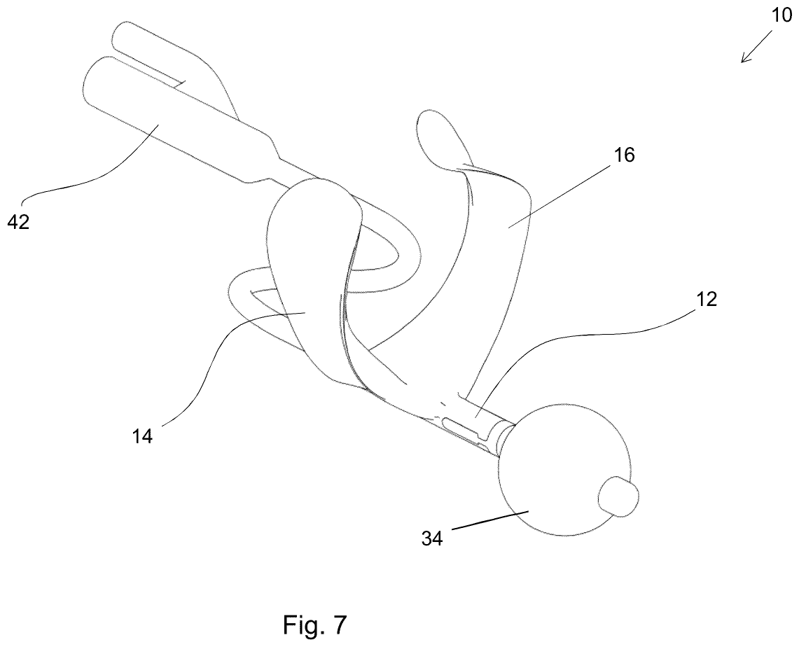

FIG. 7 illustrates a medical device including the body portion and wing portions, the balloon portion and also a pump for the balloon; and

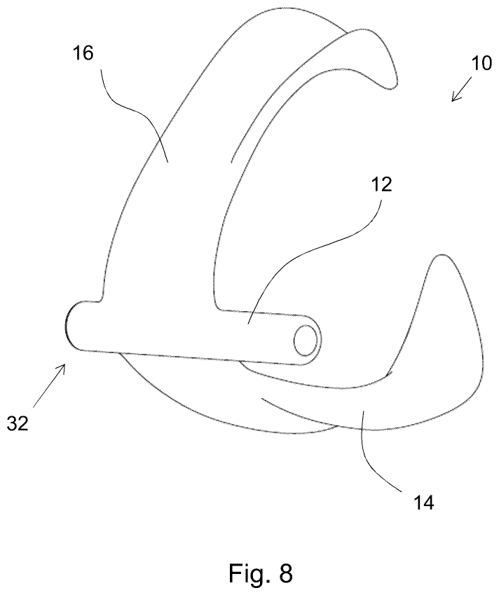

FIG. 8 is a perspective view of an alternative form of the device.

FIG. 1 shows a device 10 for spreading the labia of a female patient during a procedure such as catheterisation. The device 10 comprises a body portion 12 from with a first wing portion 14 and a second wing portion 16 extend. In use, the outer surfaces 18 of the wing portions 14, 16 act against the labia of the patient to push the labia outwardly, thereby spreading them. This allows a medical practitioner to access the body parts that would usually lie beneath the labia and be hidden, such as the urethra.

In order to use the device the medical practitioner first uses one hand to manually spread the labia, and then applies the device to the patient. The manual spreading of the labia may be done in the usual way, e.g. with fingers reaching down from above, i.e. downward in a direction toward the anus. The device then acts to hold the labia in the spread position as well as to apply a force to further spread the labia.

In order to properly position the device 10 such that the wing portions 14, 16 can interact with the labia as intended, the body portion 12 is provided with a distal section 12 for insertion within the vagina of the patient. The distal section 12 therefore provides a point of reference for a medical practitioner when initially positioning the device 10 on the patient, and thus ensures that the wing portions 14, 16 are held in the correct position and orientation during use. The body portion 12 of FIG. 1 is shown as a hollow cylindrical tube, with an opening 22 at its distal end. This opening 22 is suitable for receiving other medical instrumentation that may be used in conjunction with the device 10, as will be described further below in relation to FIGS. 5 to 7.

The wing portions 14, 16 are made of an elastic material and are elastically biased into the open position shown in FIG. 1. The material may for example by a medical grade polypropylene or polyurethane, and the wing portions 14, 16 are advantageously formed along with the body portion 12 in a single unitary piece, e.g. as shown in the Figures. In the open position, the distance between the two tips 24 of the wing portions 14, 16 (known as the wing distance, WD) is at its maximum. However, the elasticity of the wing portions 14, 16 enables them to rotate about the longitudinal axis A of the body portion to move out of their resting open position. The wing portions 14, 16 can be gripped by a user and rotated toward one another until their tips 24 are in contact, i.e. until the wing distance WD between the two tips 24 is zero. This position is known as the insertion position of the device, as this is the position in which the wing portions 14, 16 are inserted between the labia due to the area between the wing portions 14, 16 being minimised. This is done with the labia having been spread manually by the user's other hand. When the wing portions 14, 16 are correctly positioned between the labia of the patient, the wing portions 14, 16 are released by the user and rotate outwardly toward the open position into a spreading position. The spreading position is the position in which the wing portions 14, 16 lie when in use, wherein the outward elastic force of the wing portions 14, 16 is equal to the inward reaction force from the labia. As such, depending on the particular anatomy of the patient, the wing distance WD in the spreading position may be smaller than the wing distance WD in the open position, or the spreading position may be the same as the open position. The device 10 is thus adjustable depending on the particular size and/or shape of the patient's labia, further ensuring maximum comfort for the patient. Thus, the elastic deformability of the wing portions 14, 16 enables easy positioning of the device 10 on the patient, both during insertion and during spreading.

As can be seen in FIG. 1, the wing portions 14, 16 are curved both along their length, l, and along their width, w. In particular, the wing portions 14, 16, are each curved towards the body portion 12 along their length l and curved away from the body portion 12 along their width w. In other words, the inner surfaces 26 of the wing portions 14, 16 are curved convexly along their width w, and are curved concavely along their length l, whilst the outer surfaces 18 of the wing portions 14, 16 are curved concavely along their width w and convexly along their length l. This mimics the shape of the labia to ensure maximum comfort for the patient.

Turning now to FIG. 2, it can be seen more clearly how the wing portions 14, 16 curve inwardly in the lengthwise direction toward the body portion 12. This lengthwise curvature can be defined in relation to a vertical axis B of the device. In particular, it can be seen that, for each wing portion 14, 16, the angle α_{b} between the base 28 and the vertical axis B is a positive obtuse angle whilst the angle at between the tip 24 and the vertical axis B is a positive acute angle. It can be further seen from FIG. 2 that the degree of curvature of the wing portions 14, 16 along their length l is not constant. Rather, the degree of curvature increases from the bases 28 to the tips 24 of the wing portions 14, 16, i.e. the angle αₜ is closer to 90° than the angle α_{b}. This further mimics the shape of the labia and prevents the labia being pushed apart too far, thus straining them and causing discomfort for the patient.

In FIG. 2, an inflatable balloon portion 34 is shown extending from the distal end 20 of the body portion 12; this will be described in further detail below with reference to FIGS. 5 to 7.

FIG. 3 shows a clearer view of the curvature of the wing portion 14 along its width w. Although wing portion 14 is shown in FIG. 3, the same shape and curvature is present in wing portion 16. It can be seen from FIG. 3 that the degree of curvature in the width-wise direction is not constant along the length l of the wing portion 14, but rather the width-wise curvature is larger at the lengthwise midpoint M_{L} of the wing portion 14 than it is at the base 28 or the tip 24. In other words, the width-wise curvature increases from the base 28 to the lengthwise midpoint M_{L}, and then decreases from the lengthwise midpoint M_{L} to the tip 24. This form of the wing portions 14, 16 allows them to more effectively spread the labia in use. FIG. 3 further shows that the wing portion 14 is rounded at its tip 24, which prevents the the wing portions 14, 16 feeling as though they are “cutting into” a patient during use.

An alternative view of the device 10 is shown in FIG. 4. In FIG. 4, it can be seen that the body portion 12 comprises a slit 30 at its proximal section 32, the proximal section 32 being the section of the body portion 12 opposite the distal portion 22. The slit 30 is rectangular in shape and extends from the proximal end of the body portion 12 to a point backward (or aft) of the proximal edges of the wing portions 14, 16. The slit 30 enhances the flexibility of the wing portions 14, 16, thereby making it easier for a user to rotate them into the insertion position. In some embodiments, such as the one shown in FIG. 8, the device 10 does not comprise slit 30.

The device 10 may comprise additional features, such as an inflatable balloon portion 34 as shown in FIGS. 5 and 6. The inflatable balloon portion 34 comprises an attachment section 36, a central tube 38, and a flexible balloon 40. The attachment section 36 and central tube 38 are inserted within the body portion 12, as shown in FIG. 7, and at least the central tube 38 is glued to the inner walls of the body portion 12 to fixedly attach the inflatable balloon portion 34 to the device 10. The attachment section 36 and the central tube 38 are hollow in order to allow fluid to pass through the body portion 12 into the flexible balloon 40, thereby inflating the balloon 40. The balloon 40 is shown in its deflated state in FIG. 5, which is the state it is in during insertion into the vagina. FIG. 6 shows the balloon 40 in its inflated state, which occurs after the wing portions 14, 16 have been released and correctly positioned within the spreading position. As the balloon 40 expands, it contacts the walls of the vagina and forms a tight-fit relationship with the walls of the vagina, thus allowing the device 10 to be fixed in its spreading position during use.

All of the components of the inflatable balloon portion 34, including the attachment section 36 and the central tube 38, may be made of a flexible material for easy insertion within the vagina with the distal section 22 of the body portion 12. The attachment section 36 is threaded for attachment to a fluid pump 42, as shown in FIG. 7. The fluid pump 42 is inserted through body portion 12 and screwed or otherwise coupled (e.g. friction fit, bayonet fitment etc.) onto the inflatable balloon portion 34 via attachment section 36. As such, the attachment between the inflatable balloon portion 34 and the fluid pump 42 is reversible, and the fluid pump 42 can be removed once the balloon 40 is inflated and the device 10 is fixed in its spreading position. Fluid, preferably air, is pumped into the inflatable balloon portion 34 through the fluid pump 42, thereby inflating the balloon 40.

Thus, a medical practitioner wanting to perform catheterisation on a female patient 10 may do so with both hands free as the device 10 itself is acting to spread the labia/hold the labia in the spread position and thus to allow access to the urethra. To apply the device to the patient, after initially manually spreading the labia, the practitioner will firstly insert the distal section 22 with the inflatable balloon portion 34 into the vagina. The wing portions 14, 16 are then gripped and rotated toward one another into the insertion position, where the distance between the tips of the wing portions 14, 16 is minimised. The wing portions 14, 16 are located, whilst in the insertion position, between the labia of the patient. Once correctly positioned between the labia, the wing portions 14, 16 are released and rotate outwardly to push/hold the labia apart. The medical practitioner may withdraw their hand at the same time/after the device is in place. The wing portions 14, 16 will then settle into their spreading position once the outward force from the wing portions 14, 16 is matched by the internal reaction force of the labia. Once the medical practitioner is content with the final spreading position of the wing portions 14, 16, fluid is pumped into the inflatable balloon portion 34 using the fluid pump 42 until the balloon 40 is inflated such that it pushes against the walls of the vagina. The fluid pump 42 can be attached to the rest of the device 10 either once the wing portions 14, 16 are already positioned between the labia, or prior to the positioning the device 10 on the patient. Once the device 10 is secured in place on the patient, the fluid pump 42 can be detached from the rest of the device 10 and put to the side to prevent the pump 42 obstructing the procedure. When the procedure is finished, the practitioner can reattach the pump 42 to the device and use it to deflate the balloon 40. To remove the device 10, the wing portions 14, 16 can again be rotated by the practitioner toward one another and removed from the labia. The distal section 22 and inflatable balloon portion 34 can then be removed from the vagina and the device discarded.

FIG. 8 shows an embodiment of the device 10 without the slit 30 in proximal section 32 of body portion 12. In this embodiment, the device 10 is formed of a suitably flexible material for allowing the wing portions 14, 16 to be rotated toward one another through deformation. In particular, the wing portions 14, 16 and/or the body portion 12 of FIG. 8 are formed of a medical-grade polypropylene homopolymer, such as Lotte SJ-170M polypropylene.

Thus, the device 10 provides a comfortable, reliable, and “hands-free” mechanism for the labia of a patient to be spread/kept spread without requiring constant interference from a medical professional. As a result, procedures in which the labia would typically need to manually be spread throughout, such a catheterisation and the like, can be performed by a single practitioner.

1. A device for spreading the labia of a patient, the device comprising:
a body portion comprising a proximal section and a distal section, wherein the distal section of the body portion is for insertion into the vagina of the patient; and
first and second wing portions extending from the proximal section of the body portion, the first and second wing portions being configured to, in use, push against the labia and thereby spread them;
wherein the first and second wing portions are rotatable about the body portion such that the first and second wing portions can rotate toward one another during insertion between the labia.

2. The device as claimed in claim 1, wherein the first and second wing portions each comprise elastic material.

3. (canceled)

4. The device as claimed in claim 1, wherein the first and second wing portions are rotatable into an insertion position, the insertion position being a position in which a distance between tips of the first and second wing portions is at its minimum.

5. The device as claimed in claim 1, wherein each of the first and second wing portions are curved toward the body portion along their length.

6. (canceled)

7. The device as claimed in claim 5, wherein each of the first and second wing portions are curved away from the body portion along their width.

8. The device as claimed in claim 7, wherein a degree of curvature along a width of each wing portion is greatest at a lengthwise midpoint of the wing portion.

9. The device as claimed in claim 1. further comprising an inflatable balloon portion attached to the distal section of the body portion, the inflatable balloon portion being for securing the distal section of the body portion within the vagina.

10. The device as claimed in claim 9 wherein the inflatable balloon portion is insertable within the distal section of the body portion.

11. The device as claimed in claim 9, wherein the device further comprises a fluid pump coupled to the inflatable balloon portion, the fluid pump being configured to fill the inflatable balloon portion with fluid during inflation.

12. (canceled)

13. The device as claimed in claim 1, wherein the first and second wing portions are elastically biased to a position in which they, in use, push against the labia and thereby spread the labia; and
wherein the first and second wing portions are elastically deformable to an insertion position in which they are rotated about the body portion toward one another.

14. (canceled)

15. (canceled)

16. A method of spreading the labia of a patient using the device of claim 1, the method comprising:
inserting the distal section of the body portion into the vagina of the patient;
gripping and rotating the first and second wing portions toward one another;
locating the first and second wing portions between the labia; and
releasing the first and second wing portions such that the first and second wing portions rotate away from one another and push against the labia.

17. (canceled)

18. (canceled)

19. The method as claimed in claim 16, further comprising:
attaching an inflatable balloon portion to the distal section of the body portion prior to insertion of the distal section into the vagina;
inserting the inflatable balloon portion into the vagina of the patient with the distal section of the body portion; and
inflating the inflatable balloon portion to secure the distal section of the body portion within the vagina.

20. (canceled)

21. (canceled)

22. The method as claimed in claim 16, wherein the method is for performing catheterisation on a patient, and further comprises inserting a catheter into the urethra of the patient after the first and second wing portions have been released.

23. A device for spreading the labia of a patient, the device comprising:
a body portion; and
first and second wing portions extending from the body portion, the first and second wing portions each being curved along both their width and their length to conform to the shape of the labia;
wherein the first and second wing portions are elastically biased to a position in which they, in use, push against the labia and thereby spread them; and
wherein the first and second wing portions are elastically deformable to an insertion position in which they are rotated about the body portion toward one another.

24. The device as claimed in claim 23, wherein each of the first and second wing portions are curved toward the body portion along their length; and wherein each of the first and second wing portions are curved away from the body portion along their width.

25. The device as claimed in claim 24, wherein the degree of curvature along the width of each wing portion is greatest at the lengthwise midpoint of the wing portion.

26. The device as claimed in claim 24, wherein the degree of curvature along the length of each wing portion is greatest at a tip of the wing portion.

27. A method of spreading the labia of a patient using the device of claim 23, the method comprising:
rotating the first and second wing portions toward one another;
locating the first and second wing portions between the labia; and
releasing the first and second wing portions such that the wing portions return to their elastically biased position.

28. The method as claimed in claim 27, wherein the step of rotating of the first and second wing portions includes rotating the first and second wing portions until a distance between a tip of the first wing portion and a tip of the second wing portion is minimised.

29. The method as claimed in claim 27, wherein the method is for performing catheterisation on a patient, and further comprises inserting a catheter into the urethra of the patient after the first and second wing portions have been released.
